(19) United States
(12) Patent Application Publication
(10) Pub. No.: US 2014/0242575 A1
(43) Pub. Date: Aug. 28, 2014

(54) KITS AND METHODS FOR IN VITRO ANALYTE DETECTION USING PRECIPITATING COMPOUND AND POLARIZERS

(71) Applicant: SurModics, Inc., Eden Prairie, MN (US)

(72) Inventor: Gary Opperman, St. Louis Park, MN (US)

(21) Appl. No.: 14/190,367

(22) Filed: Feb. 26, 2014

Related U.S. Application Data
(60) Provisional application No. 61/769,454, filed on Feb. 26, 2013.

(51) Int. Cl. G01N 33/543

(52) U.S. Cl. CPC G01N 33/543
USPC 435/6.1; 435/7.5

(57) ABSTRACT

The invention provides inexpensive kits and methods for determination of an analyte in a sample with very high sensitivity. Analyte can be determined using an analyte binding member such as an antibody or an oligonucleotide, which can be directly or indirectly linked to an enzyme. The kit and method uses a compound that is altered in the presence of the enzyme, and that precipitates into crystalline particulates. The reaction composition with crystalline particulates is placed between two polarizers which are in an orthogonal arrangement, and the presence of particulates is observed, correlating to the analyte in the sample.

The present non-provisional Application claims the benefit of commonly owned provisional Application having Ser. No. 61/769,454, filed on Feb. 26, 2013, entitled KITS AND METHODS FOR IN VITRO ANALYTE DETECTION USING PRECIPITATING COMPOUND AND POLARIZERS, which Application is incorporated herein by reference in its entirety.

### FIELD

The invention relates to kits and methods for performing in vitro detection of an analyte in a sample.

### BACKGROUND

Research and diagnostic procedures benefit from rapid, accurate, and qualitative and/or quantitative determinations of substances (“analytes”) that are present in biological samples, such as biological tissues or fluids, at low concentrations. For example, the presence of drugs, narcotics, hormones, steroids, polypeptides, prostaglandins or infectious organisms in blood, urine, saliva, dental plaque, gingival crevicular fluid, or other biological specimens is desirably determined in an accurate and rapid fashion for suitable diagnosis or treatment.

In many cases, an analyte is identified in a sample using a compound that specifically recognizes the chemical features of the analyte. Often, monoclonal antibodies specific for one or more chemical epitopes on an analyte are used. In other cases, oligonucleotides that are specific for gene transcripts (e.g., mRNA analytes) can be used to assess gene expression in a cell-containing sample.

The complex formed between the antibody (or oligonucleotide) and analyte can be detected by a variety of known methods. The most commonly used methods employ a signal generating moiety of some type which is either already attached to the antibody/oligonucleotide, or becomes attached to the antibody/oligonucleotide through further reaction. For example, in the formation of a complex of biotin with avidin, the complex may be detected using a label on either the avidin or biotin molecule. Such a label may be a radioisotope or an enzyme conjugated to the avidin or biotin. Alternatively, the avidin-biotin complex might be detected by further reaction with a labeled molecule which is specific to either or both parts of the complex. It is commonly known to do the same with antigens and their corresponding antibodies.

During an analysis procedure, the specific binding ligand of interest (such as an antigen from an infectious agent) is often detected using colorimetric, fluorescent, or chemiluminescent signals resulting from reaction of the enzyme label with its corresponding substrate.

In many colorimetric assays, a sample containing analyte is mixed with detection reagents and a colored product is produced if the analyte is present. Analysis can be carried out for a predetermined time after the assay is started (end point analysis), or can be performed over time to assess the generation of color (kinetic analysis). The concentration of analyte in test samples can be calculated from a standard curve. Typically, these types of analysis use electronic equipment, such as fluorometers or spectrophotometers to gain the desired level of sensitivity. However, in many end-use situations such complex detection and analysis equipment is not available. Another problem associated with detection assays is that low amounts of analyte are often difficult to detect without the aid of the aforementioned electronic equipment.

### SUMMARY

Generally, the invention provides kits and methods for in vitro determination of an analyte in a sample. Analyte can be determined using an analyte binding member such as an antibody or an oligonucleotide. The antibody or oligonucleotide can be directly or indirectly linked to an enzyme. In the kit and method of the invention, a compound that is altered in the presence of the enzyme, and that precipitates into crystalline particulates is used. The reaction composition with crystalline particulates is placed between two polarizers which are in an orthogonal arrangement, and the presence of particulates is observed, correlating to the analyte in the sample.

In one embodiment the invention provides a kit for detecting an analyte in a sample. The kit comprises an enzyme; a crystal-forming compound capable of forming a crystal in the presence of the enzyme; a first light polarizer; and a second light polarizer. In exemplary embodiments, the crystal-forming compound is a crystal-forming benzidine chromogenic compound, such as 3,3′,5,5′-tetramethylbenzidine (TMB).

The enzyme in the kit can be a peroxidase or oxidase, and the kit can optionally include other components like one or more redox compounds, such peroxidase substrates, oxidase substrates, which can be used to convert the compound so that it precipitates into a crystalline particulate. Another optional component is an analyte binding member, such as an antibody or an oligonucleotide, capable of specific recognition of an analyte of interest in a biological sample. The kit can also include instructions for carrying out the assay.

In another embodiment, the invention provides a method for detecting an analyte comprising steps of: providing a reaction composition comprising an analyte, enzyme, and a crystal-forming compound; allowing the reaction composition to form crystals; placing the reaction composition between first and second light polarizing materials; transmitting light through the first light polarizing material, reaction composition with crystals, and second light polarizing material; and then observing the reaction composition by the light transmitted.

The method can include one or more optional sub-steps of: immobilizing the analyte on a substrate during the step of providing; contacting the analyte immobilized on the substrate with an analyte binding member, wherein the analyte binding member is directly coupled to an enzyme, or the method comprises one or more substeps of indirectly coupling an enzyme to an analyte binding member, to form an enzyme-immobilized substrate; and/or contacting the enzyme-immobilized substrate with a composition comprising a crystal-forming compound capable of forming crystals in the presence of the enzyme.

The kit and method of the invention provide a highly sensitive assay for the detection of an analyte in a sample without requiring use of large or expensive electronic detection equipment such as fluorometers or spectrophotometers. For example, concentrations of analyte below 8 nano molar were detectable by eye when precipitated crystals were formed and observed between the polarizers.

### BRIEF DESCRIPTION OF THE FIGURE

FIG. 1 shows images of oligonucleotide immobilized glass slides including precipitated TMB crystalline particulates visualized using a flat bed scanner, polarizing filters, and a confocal scanner.

### DETAILED DESCRIPTION

The embodiments of the present invention described herein are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention is directed to kits and methods for detecting the presence of an analyte in a sample. In experiments associated with the invention, it has been found that very low levels of analyte can be detected in a sample using a compound (such as tetramethylbenzidine) that forms crystalline particulates in the presence of an enzyme, and a pair of polarizing filters. When analyte is present in the sample, an enzyme-linked analyte binding assay can be performed to cause precipitation of the compound into crystalline particulates. The formed particulate can then be placed between the two polarizers which significantly improves visualization of the precipitated material, as opposed to unaided visualization using only the eye. Low levels of analyte were able to be detected in the sample without using expensive detection equipment such as spectrophotometers or fluorometers.

The “kit” or “system” can include components (“kit components” or “system components”) for carrying out the method of the invention. The kit includes polarizers, crystal-forming compound, and enzyme. Other components can optionally be included in the kit for carrying out analyte detection. For example, the kit can optionally include a light-transmitting reaction substrate or surface (such as a glass slide or thin polymeric film) on which the enzyme and the crystal-forming compound can be placed. The components can be packaged together and provided to a user, along with instructions for carrying out the method using the components. Chemical components can be provided in the system in dry form, or in solution, or both.

Increasing the detection sensitivity for an analyte in an analyte-binding assay, such as an ELISA, using relatively inexpensive polarizing filters instead of more expensive electronic equipment is highly desirable as it enables the end user greater flexibility in carrying out analysis.

The kit and methods of the invention use two or more polarizers, which are generally optical filters that allow passage of light of a particular polarization. Polarization refers to a specific orientation of the light wave's electric field at a point in space over one period of the oscillation. The polarizer can block or reflect waves of other polarizations. Polarizers of the invention can polarize electromagnetic waves in the visible spectrum. A “polarizer” of the current invention can also be referred to as a “polarizing filter” or “polarizing component.” Linear polarizers, such as absorptive polarizers, or beam-splitting polarizers, can be used.

Some conventional plastic polarizers are made from a synthetic polymer such as polyvinyl alcohol (PVA) containing a polarizing agent such as iodine or a hydrophilic dichroic dye. During manufacture, the material is stretched to cause the PVA chains to align, and valence electrons from the iodine dopant are able to move linearly along the polymer chains. Incident light polarized parallel to the chains is absorbed by the material, but light polarized perpendicularly to the chains is transmitted. The polarizer can be made from other thermoplastics such as polyvinylee or polyacetelyne. Other dichroic substances, such as Chloratine Fas Red, Dhrysophenine, Sirius Yellow, Bensopurpurine, Direct Fast Red, Brilliant Blue 6B, Chlorasol Black BH, Direct Blue 2B, Direct Sky Blue, Diamine Green, Congo Red and Acid Black, can be used in the polarizer.

The polarizer can also be constructed as a composite of two or more layers, one of which is a polarizing layer. Material of a composite can include other polymeric materials, such as a cellulose-based polymer like cellulose aceto butyrate (CAB).

Exemplary polarizing articles include high contrast linear polarizing films, high contrast plastic linear polarizers, linear glass polarizing filters, NIR linear polarizers, NIR linear polarizing film, visible linear polarizing film, and visible linear polarizing laminated film. Polarizers of the kit and methods of the invention can be obtained commercially, for example, from Edmund Optics, Inc. (Barrington, N.J.). Commercially available polarizers may also be modified or configured to be used with the kit and methods of the invention.

Polarizers of the kit or methods of the invention can optionally be defined by one or more properties of the polarizer such as percentage light transmission, wavelength range, extinction ratio, degree of polarization, and polarization efficiency.

Transmission (% T) is a common unit used to quantitatively express how an optical filter transmits light. Percent transmission is measured at a particular wavelength (or wavelength range) and is the ratio of transmitted light intensity (I) to incident light intensity (Io), expressed as a percentage % Tλ=I/Io*100.

Degree of Polarization (DOP) is defined as DOP=Iₚₒₗ/(Iₚₒₗ+Iᵤₙₚ) where Iₚₒₗ and Iᵤₙₚ are the intensity of polarized light and unpolarized light, respectively. When DOP=0, light is said to be unpolarized, and when DOP=1, it is totally polarized. Intermediate cases correspond to partially polarized light.

Polarization Extinction Ratio (PER) is the ratio of the minimum polarized power and the maximum polarized power, expressed in dB. Any polarization component will specify this value as a specification.

Polarizers of the kit and methods of the invention can also optionally be defined by physical properties such as thickness, surface area, shape, weight, and optional curvature.

The polarizers will have a direction of polarized light, and the direction can be known for each polarizing filter. In use, the first and second polarizers can be arranged so their directions of polarization are orthogonal to each other (i.e., they are rotated 90° from each other). In the kit, the first and second polarizers can be unattached, and the user can manually arrange the polarizers to they are in a desired orthogonal arrangement during analysis. In other arrangements, the first and second polarizers are directly or indirectly attached to each other and in an orthogonal arrangement during analysis. For example, the first and second polarizers can be mounted in a rigid or moveable frame.

The kit and methods of the invention also include a crystal-forming compound. The compound can precipitate into a crystalline particulate during the analysis method. Upon precipitation, the compound molecules become ordered (crystalline, as opposed to amorphous). An enzyme, introduced when analyte is present in the sample, promotes a chemical change of the compound leading to its precipitation. The precipitated crystals are able to rotate light and exhibit birefringence, and are highly detectable when observed between the two polarizers.

In some aspects, the crystal-forming compound becomes oxidized which leads to its precipitation and formation of crystals. For example, the crystal-forming compound can have appreciable solubility in a reaction composition, such as an aqueous composition, in a non-oxidized state, or partially oxidized state, and then upon oxidation to a higher state the compound becomes less soluble in the composition and forms precipitates of crystalline particulates.

In some aspects, the crystal-forming compound is a crystal-forming aromatic amine chromogenic compound. The crystal-forming aromatic amine chromogenic compound can include an aryl (or aryl ring system) and an amine group, wherein the amine group is bonded to an atom in the aryl ring (or system), and includes the following chemical features:

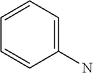

An aryl ring system can include an aryl ring fused to or more other rings, as exemplified by naphthalene, phenalene, anthracene, indacene, indene, including heterocyclic aryl ring systems such as acridine, carbozole, quinoline, indole, indolizine, and benzofuran as well. The one or more aryl rings of the crystal-forming aromatic amine chromogenic compound can be substituted (“ring substituted”) or unsubstituted. The crystal-forming aromatic amine chromogenic compound can include one or more amine group(s), at least one of which is bonded to the aryl ring.

Crystal-forming aromatic amine chromogenic compounds include “benzidine chromogenic compounds,” which refer to compounds selected from benzidine (biphenyl-4,4′-diamine), compounds having a benzidine core, and ring and/or amine group substituted derivatives thereof. The crystal-forming benzidine chromogenic compounds can precipitate to crystalline material detectable using the polarizers of the method and kit. In some embodiments, the kits and methods of the invention use a benzidine derivative such as 3,3′,5,5′-tetramethylbenzidine. Benzidine derivatives include those of formula I:

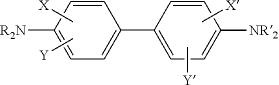

where X, X′, Y and Y′ and R and R′ are independently hydrogen, alkyl, or alkoxy, with the alkyl or alkoxy group(s) having up to six carbon atoms. More specifically, X, X′, Y and Y′ and R and R′ are independently hydrogen, alkyl, or alkoxy, with the alkyl or alkoxy group(s) containing four or less carbon atoms. See, for example, U.S. Pat. No. 6,376,252.

In the presence of horseradish peroxidase (HRP) and hydrogen peroxide, tetramethylbenzidine (3,3′,5,5′-tetramethylbenzidine; TMB) is oxidized and precipitated to a colored crystalline material. As described by Josephy et al. (1982; J. Biol. Chem. 257:3669-3675), TMB (λₘₐₓ 285) is oxidized to form oxidized TMB, a blue product (λₘₐₓ 652) which is a one-electron oxidation product, and then is further oxidized to form a yellow product (λₘₐₓ 450), which is the two-electron oxidation product. (Reaction Sequence A).

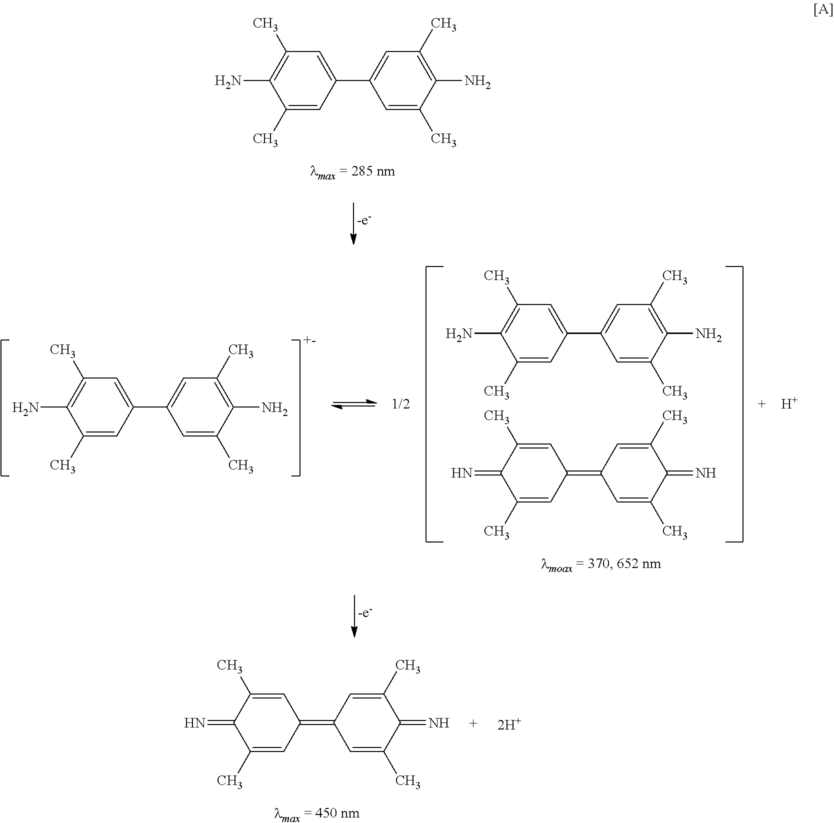

Benzidine has a solubility in water of about 0.94 g/100 mL at 100° C.

The kit can also include an enzyme for promoting the conversion of the crystal-forming compounds to a crystalline particulate. The enzyme can be, for example, a peroxidase, an oxidase, or a conjugate of a peroxidase or an oxidase.

The kit can also include reagents for the detection of an analyte and analysis of the crystals. For example, the kit can optionally include an analyte specific binding member. The analyte specific binding member can be used for the detection of an analyte of interest in a biological sample.

The kit can also optionally include an analyte positive control, an analyte negative control, or mixtures thereof.

As described herein, the components of the kit allow analysis to be carried out without necessarily using specialized equipment, and therefore use of the unaided human eye can be sufficient for detection of very low levels of analyte in a sample. However, optionally, one may choose to conduct analysis using specialized equipment known in the art to quantify and measure the wavelength and/or absorbance of the sample. Such equipment includes, but is not limited to UV-visible spectrophotometers, multiwell plate readers, CCD cameras, and optical scanners.

Generally, analysis can be performed to determine the presence and/or amount of an analyte in a sample. The term “analyte” refers to any substance or chemical constituent of a sample that is being analyzed. The analyte can be a natural compound, such as one that is produced by an organism, or can be a non-natural compound, such as a synthetic compound used as a bio-affecting agent like a drug, a pesticide, or an herbicide. Methods, compositions and kits of the invention can be used for the determination of analytes in numerous industries including, but not limited to, health care, food manufacture and processing, chemical analysis and production, agriculture, environmental control, and the like. In some aspects, an ELISA (enzyme-linked immunosorbent assay) is used for detection of an analyte, and the kit and method, along with the crystal-forming compound and polarizers, includes components for carrying out an ELISA.

Various types of analytes can be detected and quantified in a sample using the methods of the invention. In some aspects, detection of the analyte is facilitated by using an analyte binding member, such as an antibody. In other aspects, methods of the invention provide for detection of an analyte without utilizing an analyte binding member. Non-limiting exemplary analytes include drugs, drug metabolites, biomarkers, hormones, antibiotics, food supplements, food additives, naturally occurring contaminants, dyes, microorganisms and their toxins, fungi, viruses, pesticides, herbicides, organic components of waste discharges, tissue specific markers, tissue specific enzymes, cytokines, chemokines, growth factors, receptor ligands, enzymes, nucleic acids, lipids, and small organic molecules, such as glucose and peroxides.

For example, in the food processing and manufacturing industries for humans, as well as domesticated and farm animals, ELISAs are used for the detection of various analytes that are polypeptides, such as soy proteins and gluten, which can be allergens, and other compounds such as antibiotics and hormones.

As other non-limiting examples, in the health care industry ELISAs are used for the detection of various analytes in blood, urine, and other body fluids. Detection of analytes such as erythropoietin (EPO), adrenocorticotropic hormone (ACTH), calcitonin, parathyroid hormone (PTH), thyroid stimulating hormone (TSH), prostate-specific antigen (PSA), human chorionic gonadotropin (HCG), follicle stimulating hormone (FSH), and growth hormone (GH) can be performed.

The sample including an analyte to be detected can be a biological or a non-biological sample.

A biological sample can be any material taken from an organism such as body fluid from a mammal, material derived from an organism, or a sample that has organisms in it. Biological samples include certain tissues, or body fluid such as blood, sputum, urine, saliva, mucus, vitreal fluid, synovial fluid, semen, cerebrospinal fluid, bone marrow, amniotic fluid, bile, sweat, etc. Biological samples can be obtained from patients and analyzed for the absence or presence of analytes associated with disease states. Methods and kits of the invention can detect an analyte in a sample, and such information can be used to determine the absence, presence or degree of a disease state. Biological samples can also include sections of tissues such as frozen sections taken for histological purposes which can also be analyzed for analytes associated with disease states.

Other biological samples can be those derived from fermentation, cell culturing, bio-fuel production, wastewater treatment, and agriculture. These include food products such as milk, wine, beer, and the like; chemical streams, or waste streams from chemical plants, rivers, and the like. Where the sample is initially complex, solid, or viscous, it may need to be extracted, dissolved or diluted in order to obtain a sample having the appropriate characteristics for use in the immunoassay.

An example of a non-biological sample is a composition of a chemically-synthesized component, such as a synthetic drug for human treatment or a synthetic pesticide for agricultural use.

Analyte detection can be performed using the components of the kit.

The kit and method can include a compound having specific affinity for the analyte. A compound with specific affinity is referred to herein as an “analyte binding member” or “analyte binding moiety,” which refers to any sort of chemical group that can bind or interact with the analyte. The binding moiety can include naturally occurring molecules or derivatives of naturally occurring molecules, or synthetic molecules, such as small organic molecules, or larger synthetically prepared molecules, such as polymers. Examples of binding moieties include polypeptides, nucleic acids, polysaccharides, and portions of these types of molecules that can bind a target species. Nucleic acids such as oligonucleotides that have a length sufficient to undergo complimentary hybridization to a target nucleic acid analyte in a sample can be used as the analyte binding moiety.

The binding moiety can be an antibody, which is a protein that recognizes a particular epitope on the analyte. In this regard, the analyte may also be referred to as an “antigen” as it is common for antibody-antigen interactions to be described. An antibody can be a polyclonal antibody, a monoclonal antibody or a genetically engineered molecule capable of binding the corresponding member of a specific binding pair. One class of polypeptides that can be used as a binding moiety in the invention includes antibodies and antibody fragments.

Antibody and antibody fragments having specificity towards desired analytes are commercially available or can be prepared by techniques known in the art. For example, monoclonal antibodies (mAbs) can be obtained by any technique that provides for the production of antibody molecules by continuous cell lines in culture. These include, for example, the hybridoma technique (Kohler and Milstein, Nature, 256:495-497 (1975)); the human B-cell hybridoma technique (Kosbor et al., Immunology Today, 4:72 (1983); and the EBV-hybridoma technique (Cole et al., Monoclonal Antibodies and Cancer Therapy, Alan R. Liss, Inc., pp. 77-96 (1985)). Such antibodies may be of any immunoglobulin class including IgG, IgM, IgE, IgA, IgD and any subclass thereof.

Fab or Fab′2 fragments can be generated from monoclonal antibodies by standard techniques involving papain or pepsin digestion, respectively. Kits for the generation of Fab or Fab′2 fragments are commercially available from, for example, Pierce Chemical (Rockford, Ill.).

In some cases, the binding moiety is conjugated to another compound which facilitates the precipitation of the crystal-forming compound, such as described herein.

In some aspects of the invention, the method or kit of the invention includes an oxidase or a peroxidase. As a general matter, an oxidase or a peroxidase can be used to generate a radical resulting in the transfer of electrons from one component to another. For example, in some cases when the crystal-forming compound is present, it becomes oxidized to a product which precipitates out of the reaction solution in the form of crystalline particulates.

In some cases the oxidase or peroxidase is coupled to a binding moiety for the detection of an analyte. In these cases, the oxidase or peroxidase can simply serve to cause reduction of a peroxide compound, such as hydrogen peroxide, providing a chemical environment for the oxidation of the crystal-forming compound to cause its precipitation. Commercially available peroxidase conjugates, such as those described herein, can be used.

In other cases the oxidase or peroxidase has specificity towards an analyte of interest in the biological sample. For example, glucose oxidase (GOx) (EC 1.1.3.4) is an oxido-reductase that catalyses the oxidation of glucose to hydrogen peroxide and D-glucono-δ-lactone. In order to quantitate the amount of glucose in a biological sample, glucose oxidase and a peroxidase, such as horseradish peroxidase, can be added directly to the sample. Glucose oxidase, which causes the formation of hydrogen peroxide, can be reduced to provide conditions resulting in the oxidation of the crystal-forming compound. Xanthine oxidase is another enzyme that generates hydrogen peroxide by using hypoxanthine and xanthine as substrates. L-gulonolactone oxidase (EC 1.1.3.8) catalyzes the reaction of D-glucuronolactone to L-xylo-hex-3-gulonolactone and hydrogen peroxide. Using this type of approach, the analyte or the analyte binding member may not have be immobilized on a solid surface, such as performed using ELISA. Other oxidases include galactose oxidase, hexose oxidase, and pyranose oxidase.

Peroxidase enzymes useful for the methods of the invention can be obtained from a variety of sources, such as plants. Common, commercially available peroxidases are from horseradish and soybean. Horseradish peroxidase has an approximate molecular weight of 44 kDa, and is a single chain polypeptide glycoprotein with disulfide bridges that includes hemin plus Ca²⁺. HRP specific activity can be expressed in pyrogallol units (one pyrogallol unit will form 1.0 mg purpurogallin from pyrogallol in 20 sec at pH 6.0 at 20° C.) or ABTS units (one ABTS unit will oxidize 1 μmole of ABTS per minute at 25° C. at pH 5.0). HRP can be inhibited by sodium azide, L-cystine, dichromate, ethylenethiourea, hydroxylamine, sulfide, p-aminobenzoic acid, Cd⁺², Co⁺², Cu⁺², Fe⁺³, Mn⁺², Ni⁺², and Pb⁺².

Other peroxidases include lactoperoxidase, microperoxidase, NADH peroxidase NADPH peroxidase, fatty-acid peroxidase, and catalase.

In some modes of practice, a “peroxidase conjugate” is used in the methods for detecting an analyte. A conjugate generally refers to a compound that comprises two substances, wherein one of the substances is coupled to the other. Coupling of the conjugate can be covalent or non-covalent. A peroxidase conjugate can be one where a peroxidase enzyme is coupled to a binding moiety that detects the analyte, or that detects the binding moiety that detects the analyte. Exemplary peroxidase conjugates that can be commercially obtained include avidin-peroxidase conjugates, monoclonal anti-FLAG™ M2-peroxidase conjugates, anti-glutathione-S-transferase(GST)-peroxidase conjugates, anti-mouse IgG-peroxidase conjugates, anti-goat/sheep IgG-peroxidase conjugates, Protein G-peroxidase conjugates, Protein-A peroxidase conjugates, anti-rabbit IgG-peroxidase conjugates, and strepavidin-peroxidase conjugates, from, for example, Sigma-Aldrich.

In some cases, it is not necessary to identify the analyte (to be detected) in the biological sample with an analyte binding member, such as an antibody. Some assays use enzymes to measure biological substances in samples. For example, some assays use a peroxidase enzyme to measure peroxides present in a sample. Conversely, some assays detect the presence of biological enzymes by using the enzyme's corresponding substrate. For example addition of hydrogen peroxide to a sample can be used to test for peroxidase enzymatic activity.

In some modes of practice the kit or method uses an enzyme-linked immunosorbent assay (ELISA). Generally, an ELISA uses a solid-phase immunoassay to detect the presence of an analyte in a liquid sample. Various ELISA formats are known in the art, and any of these can be used in conjunction with the crystal forming compound and polarizers. Although antibodies are typically used in ELISAs, any sort of analyte-binding member can replace the antibody to provide analyte specific interaction, such as oligonucleotides. As such, methods of the invention can be used with any enzyme-linked solid phase analyte binding assay. The methods of the invention can include any analyte-binding reagent immobilized on the solid phase, along with the enzyme and crystal forming compound. The invention is not limited to any type of analyte binding assay, or any type of analyte binding member, but some examples are discussed to illustrate aspects of the invention. The particular immunoassay format employed will depend on the particular analyte characteristics, the sample characteristics, the available reagents, and the like.

One example of a common ELISA format is the “direct, antigen down” ELISA which is often used when the biological substance measured is antibody. In this format a purified protein (the antigen) is absorbed or covalently bonded to a plastic or glass surface. The plastic or glass surface can be configured to be placed between the two polarizers. The plastic surface can be a thin plastic material, or even a film that can adhere to a surface of one of the polarizers. Optionally, the antigen is absorbed or covalently bonded to a plastic or glass surface of the polarizer.

The plastic or glass surface can be washed to remove excess antigen and then blocked with a blocking agent that can be protein-based or synthetic, to prevent non-specific binding of the antibody. Next, a sample is added to the well that includes antibodies that specifically bind the antigen immobilized on the plastic or glass surface. Excess sample is then washed off. After washing, secondary antibody conjugated to a peroxidase enzyme specific for the animal antibody in the biological sample is added. For example, if the sample is human, an anti-human antibody (e.g., an anti-human IgG) is used for detection. Formation of the crystals can be formed by adding a reactant, such as hydrogen peroxide, for the peroxidase along with the crystal-forming compound, such as a crystal-forming aromatic amine compound like TMB. After the reaction proceeds for a desired period of time, the sample can be placed between the two polarizers, light can be transmitted from through the polarizers and the sample, and the presence of any crystals as a result of the presence of the analyte, can be observed.

In other cases, for example, a “sandwich” ELISA can be performed by first immobilizing the analyte binding member (e.g., an analyte “capture” antibody) on the plastic or glass surface. The plastic or glass surface is then typically blocked with a non-specific protein to prevent adherence of components from the biological sample when added in a next step. A biological sample containing the analyte is placed on the area of plastic or glass having the immobilized antibody, and the analyte specifically interacts with the antibody. The surface is washed and then a solution of a second analyte binding member (such as an antibody conjugated to a peroxidase enzyme, called a detection antibody) is added which interacts with the analyte already immobilized by the plastic or glass bound antibody. The analyte therefore effectively becomes “sandwiched” between the antibody absorbed on or bonded to the plastic or glass, and the enzyme-conjugated antibody. A precipitation reaction can then be performed by adding a reactant, such as hydrogen peroxide, for the peroxidase along with the crystal-forming compound.

Alternatively, the “sandwich” approach can be performed by first mixing an enzyme-conjugated antibody with a biological sample having an analyte. The enzyme-conjugated antibody is used in excess and analyte-enzyme-conjugated antibody complex forms in the sample. The sample is then transferred to a plastic or glass surface having an analyte capture antibody immobilized thereon. The analyte capture antibody binds the analyte-enzyme-conjugated antibody complex, and then the well can be washed to remove non-bound material. A crystal-forming precipitation reaction can then be performed using the crystal-forming compound.

Typically, steps for the immobilization of the analyte or analyte binding member (e.g., antibody), binding of analyte binding member to the analyte, and subsequent binding of the analyte-analyte binding member complex can be performed using a suitable solutions, such as incubation, blocking, and washing buffers. The solution can be an aqueous buffered solution, to maintain absorption of the analyte and/or analyte binding member on the plastic or glass surface, and maintain protein configuration for proper binding and enzymatic activities. The blocking solution can include a non-specific protein such as bovine serum albumin, which can effectively block vessel binding sites that remain following initial coating steps in ELISA procedures (e.g. 5% BSA-PBS). Washing buffers can include a surfactant such as Tween (an exemplary washing buffer, PBS-T, contains 10 mM phosphate buffer pH 7.4, 150 mM NaCl, and 0.05% Tween 20).

In some embodiments, the systems and method of the invention is configured for detection of a nucleic acid analyte. Any type of nucleic acid capable of interacting with a complimentary nucleic acid sequence can be detected as the analyte. Exemplary analytes include nucleic acid fragments (e.g., restriction digested DNA) of bacterial, viral, and eukaryotic DNA, including genomic DNA, plastid DNA, mitochondrial DNA, etc.; as well as mRNA (messenger RNA), iRNA (immune ribonucleic acid), ribozymes, siRNA (small interfering RNA), miRNA (micro RNA), and shRNA (short hairpin RNA).

In some embodiments, a nucleic acid having a sequence capable of hybridizing with a nucleic acid analyte under stringent conditions is immobilized on a plastic or glass surface. Nucleic acid from a sample, such as a biological sample, can be placed in contact with the surface with immobilized nucleic acid. Nucleic acid analyte can be present from a cell sample that is treated to disrupt the cells and optionally enrich the analyte nucleic acid. Analyte nucleic acid can undergo hybridization by complimentary bonding to the immobilized nucleic acid. Stringent hybridization conditions are well known in the art (see for example, in Sambrook et al., Molecular Cloning: A Laboratory Manual, Second Edition, Cold Spring Harbor Laboratory, Cold Spring Harbor, N.Y., (1989)). Presence of the analyte can then be detected with a free probe (non-immobilized) nucleic acid that can hybridize to the nucleic acid analyte and that is either directly attached to an enzyme, or that can be indirectly coupled to an enzyme.

In other embodiments nucleic acid from a sample is immobilized on a glass or plastic substrate, and then presence of the analyte can be detected with a free probe (non-immobilized) nucleic acid that can hybridize to the nucleic acid analyte and that is either directly attached to an enzyme, or that can be indirectly coupled to an enzyme.

In some modes of practice, an article is provided that has an amount of peroxidase immobilized on a solid surface. The peroxidase can be immobilized by its conjugation to a binding member, with the binding member directly or indirectly bound to an analyte previously immobilized on the vessel surface, using either an analyte binding member, or by the analyte being absorbed directly on the surface of the vessel. The amount of immobilized peroxidase can correlate with the amount of immobilized analyte, which correlates with the amount of analyte present in a sample. In control areas of the substrate, standards with known amounts of analyte can be used.

The system and method of the invention can be particularly useful for determining very small amounts of analyte in a sample without relying on complex electronic equipment such as spectrophotometers of fluorimeters. In some modes of practice analyte amounts of less than 8 nmol, 7 nmol or less, 6 nmol or less, 5 nmol or less, 4 nmol or less, 3 nmol or less, or 2 nmol or less are detectable using the methods and kit of the invention

In order to explain modes of practicing of the invention, in exemplary embodiments, analyte immobilization results in the corresponding immobilization of picrogram quantities of a peroxidase enzyme, such as horseradish peroxidase, on a plastic surface, such as an amount in the range of about 0.1 pg to about 100 ng (100000 pg), about 1 pg to about 10 ng (10000 pg), or about 1 pg to about 1 ng (1000 pg). In this case, the peroxidase enzyme is immobilized prior to adding the crystal forming compound.

As a general matter, a peroxide substrate is added to the peroxidase, which is immobilized on a surface of the vessel, or present in solution in the vessel. A cost-effective substrate for carrying out peroxidase reactions is hydrogen peroxide. HRP combines with hydrogen peroxide (H₂O₂) and can carry out heterolytic cleavage of the H₂O₂ oxygen-oxygen bond. The complex can oxidize crystal-forming aromatic amine substrate, such as TMB.

Hydrogen peroxide and the crystal-forming compound can be present in a reaction solution that can be contacted to the substrate with the immobilized peroxidase enzyme. The reaction solution can be prepared from components of a kit that include the compound, hydrogen peroxide, and reaction buffer. In some embodiments the hydrogen peroxide and compound are supplied in a ready-to-use one component kit. In other embodiments the compound and hydrogen peroxide are provided to a user separately, in a kit. For example, hydrogen peroxide can be provided as a concentrated stock solution (e.g., ˜2-3%). The concentrated hydrogen peroxide can be diluted in the reaction solution to exemplary amounts in the range of about 0.1 mM to about 50 mM (˜3×10⁻⁴% to ˜0.17%), or about 0.5 mM to about 10 mM (˜0.0017% to 0.034%). The kit can also include a reaction buffer, such as sodium phosphate (pH 7-8) provided in dry or concentrated form. For example, the buffer can be supplied as a 5× or 10× concentrate which can then be diluted in the reaction composition to a working concentration, such as in the range of about 10 mM to about 75 mM.

The working concentration of the crystal-forming compound can be chosen based on the type of compound, the amount of hydrogen peroxide, and/or the amount of analyte (and corresponding peroxidase) in the sample being analyzed. Exemplary ranges of the crystal-forming compound are from about 0.5 μM to about 10 mM, from about 10 μM to about 5 mM, or about 100 μM to about 3 mM, as described herein.

The precipitation reaction resulting in the formation of crystalline particulates can be carried out for a desired period of time at a desired temperature. The reaction can be visually monitored using the polarizers to determine the formation of the precipitates and presence of analyte. The incubation step will typically occur at room temperature, although a temperature in the range of about 10° C. to about 50° C. can be employed. Incubation times will typically range from about 1 to about 60 minutes, or more usually about 5 to about 45 minutes.

In order to describe aspects of the invention, 3,3′,5,5′-tetramethylbenzidine (TMB) is used as the crystal-forming compound in the presence of an immobilized HRP enzyme (associated with the analyte), and hydrogen peroxide. TMB, in the presence of HRP and H₂O₂ is oxidized and forms crystalline particulates having a blue color (λₘₐₓ 652).

In some modes of practice, analysis is performed under “stopped” conditions. By this it is meant that the reaction is allowed to proceed for a predetermined period and then terminated with a stop reagent. As such, optionally, a stop reagent can be added when the reaction reaches a desired detectability level. Progression of the reaction and formation of precipitated crystals can be monitored using the polarizers.

In practice, a glass or plastic substrate with precipitated crystalline particulates formed from the enzymatic reaction is placed between first and second polarizers. The polarizers will polarize light in a specific direction, and this can be known for each polarizing filters. In use, the first and second polarizers can be arranged so their directions of polarization are orthogonal to each other (i.e., they are rotated 90° from each other). Visible light can be provided from one side of the arrangement, through the first polarizer, precipitated sample, and the second polarizer, to provide a “dark field” image with the crystalline precipitates appearing as light particles in the dark field.

### EXAMPLE 1

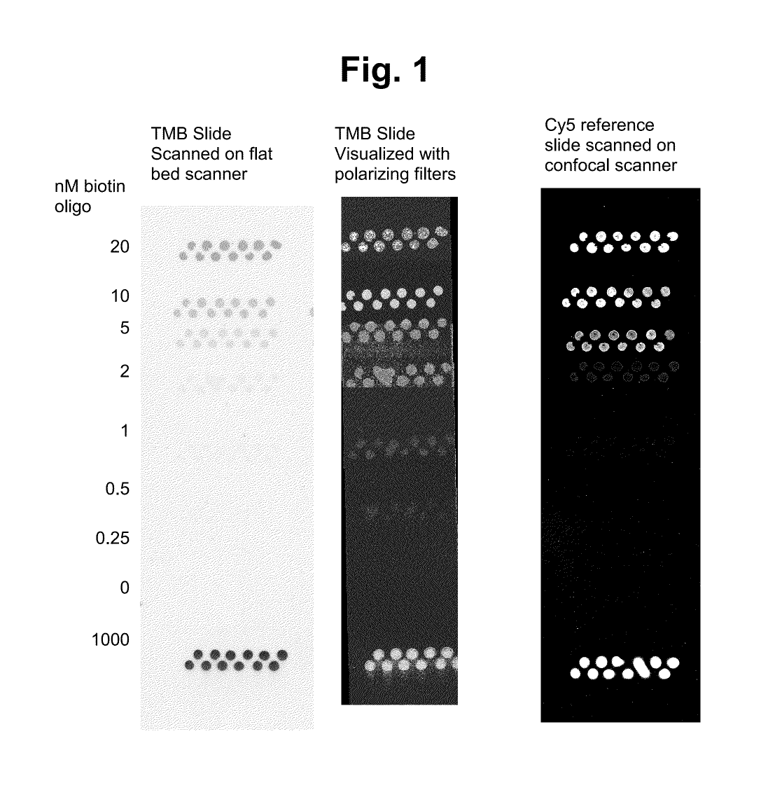

Dilutions of a biotinylated oligo with a non-biotinylated oligo (total oligo=20 μM in all conditions) were printed on activated slides. The slides were allowed to react overnight at 75% humidity. Slides were then blocked with 50 mM ethanolamine in 0.1 M TRIS, pH 9.0 for 30 minutes, washed with water 2 times and dried by spinning in staining racks in a clinical centrifuge. To visualize the printed spots slides were incubated for 30 minutes with either streptavidin-HRP (Jackson Labs) or streptavidin-Cy5 (GE-Amersham) at 1 ug/mL in PBS with 0.05% Tween-20. Slides were then washed 3 times with PBS-Tween and twice with water before visualization. The slide incubated with streptavidin-Cy5 was scanned on an Axon 4000B scanner (Molecular Devices). The slide that had been incubated with streptavidin HRP was developed by adding green precipitating TMB substrate. The slide was imaged by scanning on a flat bed scanner before being imaged using polarizing film. A polarizing film was placed on each side of the slide in a perpendicular orientation and the slide was illuminated from the bottom. Results are shown in FIG. 1.

What is claimed is:

1. A kit for detecting an analyte comprising:
an enzyme;
a crystal-forming compound capable of forming a crystal in the presence of the enzyme;
a first light polarizer; and
a second light polarizer.

2. The kit of claim 1 comprising an analyte specific binding member.

3. The kit of claim 2 wherein the analyte specific binding member is an antibody or an oligonucleotide.

4. The kit of claim 1 further comprising a light-transmitting reaction substrate or surface on which a composition comprising the enzyme and the crystal-forming compound can be placed.

5. The kit of claim 4 wherein the light-transmitting reaction substrate or surface is a glass slide or a thin polymeric film.

6. The kit of claim 4 wherein the light-transmitting reaction substrate or surface comprises an analyte binding member immobilized on its surface.

7. The kit of claim 1 wherein the first, second, or both light polarizer(s) is an absorptive polarizer(s).

8. The kit of claim 1 further comprising a light source.

9. The kit of claim 1 wherein the crystal-forming compound is a crystal-forming aromatic amine chromogenic compound.

10. The kit of claim 9 wherein the crystal-forming compound is a compound of formula I: 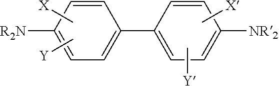
where X, X′, Y and Y′ and R and R′ are independently selected from hydrogen, C1-C6 alkyl, and C1-C6 alkoxy.

11. The kit of claim 10 wherein the compound is 3,3′,5,5′ tetramethylbenzidine (3,3′,5,5′ TMB).

12. The kit of claim 1 wherein the crystal-forming compound is supplied in the kit in an aqueous solution having a concentration in the range of 0.2 mmol to 5.0 mmol.

13. The kit of claim 1 wherein the enzyme comprises a peroxidase, an oxidase, or a conjugate of a peroxidase or an oxidase.

14. The kit of claim 1 further comprising an analyte positive control, an analyte negative control, or mixtures thereof.

15. A method for detecting an analyte comprising steps of:
providing a reaction composition comprising an analyte, enzyme, and a crystal-forming compound;
allowing the reaction composition to form crystals;
placing the reaction composition between first and second light polarizing materials;
transmitting light through the first light polarizing material, reaction composition with crystals, and second light polarizing material; and
observing the reaction composition by the light transmitted.

16. The method of claim 15 where in the step of providing, the analyte is immobilized on a substrate.

17. The method of claim 16 where in the step of providing, the analyte is immobilized on a substrate by contacting a sample containing the analyte with a surface comprising an immobilized analyte binding member.

18. The method of claim 17 where the immobilized analyte binding member is an antibody or nucleic acid.

19. The method of claim 16 comprising a step of contacting the analyte, which is immobilized on the substrate, with an analyte binding member, wherein the analyte binding member is directly coupled to the enzyme, or the method comprises one or more steps of indirectly coupling an enzyme to an analyte binding member, to form an enzyme-coupled analyte.

20. The method of claim 19 comprising contacting the enzyme-coupled analyte with a composition comprising the crystal forming compound in order to form crystals.
